(19) United States
(12) Patent Application Publication
(10) Pub. No.: US 2010/0174172 A1
(43) Pub. Date: Jul. 8, 2010

(54) MRI SYSTEM FOR UPRIGHT RADIOTHERAPY

(76) Inventor: Moshe Ein-Gal, Ramat Hasharon (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 12/349,625

(22) Filed: Jan. 7, 2009

(51) Int. Cl. A61B 5/055; G01R 33/48

(52) U.S. Cl. 600/411; 324/318

(57) ABSTRACT

An image-guided radiotherapy system including a magnet assembly operable to produce a horizontal imaging field in an imaging region, and a non-imaging field in a non-imaging region, a positioner operable to rotate an object in the imaging region about a generally vertical rotational axis, a magnetic resonance (MR) imager in communication with the horizontal imaging field, a collimator operable to collimate a generally horizontal radiation beam directed towards the object, and a radiation source operable to produce a radiation beam of charged particles substantially parallel to the non-imaging field in the non-imaging region.

### FIELD OF THE INVENTION

The present invention generally relates to radiotherapy systems, and more particularly to a radiotherapy system for irradiating an upright patient that includes an MRI imaging system, such as for real time imaging and real time target localization.

### BACKGROUND OF THE INVENTION

Image guided radiotherapy requires a determination of target location and target registration relative to a radiation beam which is typically collimated by a multi-leaf collimator. Target localization takes place for each treatment fraction in order to ensure correct registration and to adapt the treatment if needed. Real time target localization is required for tracking moving targets, e.g., the lungs. Real time imaging is used with or without implanted markers. Doing away with marker implantation by using continuous fluoroscopy or ultrasonic imaging is not recommended due to added radiation or low precision, respectively. Magnetic Resonance Imaging (MRI) has the potential to be advantageously used for this purpose due to the associated excellent soft tissue contrast sensitivity and lack of ionizing radiation. However, a conventional MRI magnetic field may interfere with the radiation beam production when the associated devices are placed in close proximity. This is due, among other things, to using charged particles, e.g., electrons, for the radiation beam production, when the particles travel along specific trajectories. A magnetic field applied to the traveling electrons, in a direction perpendicular to the electrons velocity, distorts the trajectories and thus prevents an effective beam production.

Although the impact of gradient and RF fields on charged particle trajectories can be practically controlled via magnetic shielding, the main magnetic field is difficult to shield, especially when a short distance between the radiation source and the patient is required.

### SUMMARY OF THE INVENTION

The present invention seeks to provide an improved radiotherapy system for irradiating an upright patient that includes an MRI imaging system, such as for real time imaging and real time target localization, as is described more in detail hereinbelow.

In accordance with an embodiment of the invention, a device and method are provided for substantially eliminating interference of the main magnetic field with the moving particle trajectories by aligning the magnetic field and the trajectories. By eliminating the perpendicular component, no force is applied to the charged particles by the magnetic field. Although mounting the magnet on a radiation source rotating about a recumbent patient is theoretically possible, it is far from being a simple engineering task. In accordance with an embodiment of the invention, the system is oriented toward imaging an upright patient, which is also the treatment position. A turntable for supporting the patient causes relative rotation between the radiation beam and the patient.

There is thus provided in accordance with an embodiment of the present invention an image-guided radiotherapy system including a magnet assembly operable to produce a horizontal imaging field in an imaging region, and a non-imaging field in a non-imaging region, a positioner operable to rotate an object in the imaging region about a generally vertical rotational axis, a magnetic resonance (MR) imager in communication with the horizontal imaging field, a collimator operable to collimate a generally horizontal radiation beam directed towards the object, and a radiation source operable to produce a radiation beam by moving charged particles substantially parallel to the non-imaging field in the non-imaging region. (It is noted that the beam itself can be a stream of the charged particles, but the beam can also be produced by accelerating charged particles (e.g., electrons in a LINAC) wherein the accelerated charged particles impinge on a metallic target to produce a radiation beam. In such a case, once the beam is formed the magnetic field has no effect on it; rather the effect is on the charged particles prior to impinging on the metallic target.) The magnet assembly may include shimming magnets for aligning the non-imaging field with motion of the charged particles. The radiation source may be a linear accelerator.

A collimation controller may be in communication with the collimator and the MR imager, and the collimator may be operable to dynamically shape the radiation beam.

A position controller may be in communication with the positioner and the MR imager.

The MR imager may be operable to reconstruct a 3D image from 2D projections of the object rotated by the positioner.

There is also provided in accordance with an embodiment of the present invention a method for image-guided radiotherapy including using a magnet assembly to produce a horizontal imaging field in an imaging region, and a non-imaging field in a non-imaging region, rotating an object in the imaging region about a generally vertical rotational axis, collimating a generally horizontal radiation beam of charged particles directed towards the object, the charged particles being substantially parallel to the non-imaging field in the non-imaging region, and producing MR images of the object making use of the horizontal imaging field produced by the magnet assembly.

### BRIEF DESCRIPTION OF THE DRAWINGS

The present invention will be understood and appreciated more fully from the following detailed description, taken in conjunction with the drawing in which:

FIG. 1 is a simplified illustration of an image-guided radiotherapy system, constructed and operative in accordance with an embodiment of the present invention.

### DETAILED DESCRIPTION OF EMBODIMENTS

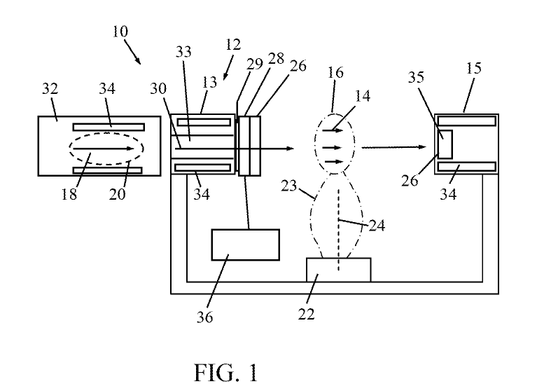

Reference is now made to FIG. 1, which illustrates an image-guided radiotherapy system 10, constructed and operative in accordance with a non-limiting embodiment of the present invention.

Image-guided radiotherapy system 10 includes a magnet assembly 12 having main magnet poles or assemblies 13 and 15 (such as two superconductive coil assemblies of the open magnet type described below). Magnet assembly 12 produces a horizontal imaging field 14 in an imaging region 16, and a non-imaging field 18 in a non-imaging region 20. A positioner 22 rotates an object 23 (such as a patient or a portion thereof) in the imaging region 16 about a generally vertical rotational axis 24. An MR (magnetic resonance) imager 26 is in communication with the horizontal imaging field 14. As is well known in the art, MR imager 26 may include an RF coil assembly (not shown) for generating a radiofrequency magnetic field pulse to the object 23 and to receive MRI information back from object 23, and a gradient coil assembly (not shown) that generates time-dependent gradient magnetic field pulses, and other processing equipment for processing and displaying the MR images.

In accordance with an embodiment of the invention, MR imager 26 can reconstruct a 3D image from 2D projections of the rotating object 23 at a high rate. Instead of acquiring three-dimensional images, MR imager 26 acquires two-dimensional projections of the rotating patient (object 23), e.g., in a direction along the main magnetic field. Each projection corresponds to a different patient orientation. Acquiring a sequence of such projections may be used in conjunction with a CT-like algorithm for reconstructing a three-dimensional image, as is known in the art. When the projections are in the beam direction, each image corresponds to a beam-eye-view projection. The pixels of the two-dimensional image correspond to line integrals of the three dimensional image, wherein the integration is along lines parallel to the central radiation beam, i.e., lines perpendicular to the beam eye-view plane. A target projection may then be localized in the beam-eye-view image and the target localization data may be used for target/beam registration.

A collimator 28, such as a multi-leaf multiple layer collimator (e.g., as described in U.S. Pat. No. 6,526,123 to Ein-Gal, the disclosure of which is incorporated herein by reference) collimates a generally horizontal radiation beam 30 in the direction of the object 23. The radiation beam 30 is produced by a radiation source 32 capable of moving charged particles (e.g., electrons, photons or others) in parallel to the non-imaging field 18 in the non-imaging region 20. The magnet assembly 12 includes a passageway 33 for the radiation beam 30 to pass therethrough. Radiation source 32 may be, without limitation, a LINAC (linear accelerator). Appropriate shielding surrounds the accelerating tube, such as a ferromagnetic material, to provide radiation and magnetic shielding. Collimator 28, such as a multi-leaf collimator, dynamically shapes the radiation beam aperture and also steers the radiation beam 30 toward the target to be irradiated, whenever the target is slightly off the desired target location. A radiation beam modulator 29, such as but not limited to, a flattening filter, may also be provided to spatially modulate the radiation beam intensity. After passing through object 23, the radiation beam 30 is detected by one or more radiation beam detectors 35.

It is noted that the main magnet assembly 13 may be part of the collimator housing, thus providing a potential reduction of the inter-poles magnet gap. The main magnet assembly 15 may be part of the housing for radiation beam detector 35.

Magnet assembly 12 may be of any type typically used in MRI systems. For example, magnet assembly 12 may employ superconductive or other type magnets, such as liquid-helium cooled and cryocooler-cooled superconductive magnets. For a helium-cooled magnet, the magnet assembly may include a superconductive main coil which is at least partially immersed in liquid helium contained in a helium Dewar which is surrounded by a dual thermal shield which is surrounded by a vacuum enclosure. For a cryocooler-cooled magnet, the superconductive main coil may be surrounded by a thermal shield which is surrounded by a vacuum enclosure. Niobium-titanium (Nb—Ti) superconductive coils typically operate at a temperature of generally 4 K, and niobium-tin (Nb—Sn) superconductive coils typically operate at a temperature of generally 10 K.

For some liquid-helium cooled superconductive magnets, the superconductive coils may be of the cryostable (non-impregnated) type having superconductive windings generally completely contacted by the liquid helium typically through porous spiral-wound electrical insulation. For other liquid-helium cooled superconductive magnets, the superconductive coils may be of the impregnated type (e.g., epoxy) having superconductive windings cooled by internal thermal conduction through the epoxy as well as along the length of the superconductor.

Magnet assembly 12 is illustrated as an open magnet system, such as described in U.S. Pat. No. 5,999,075 the disclosure of which is incorporated herein by reference, but can also be of the closed type (with a sufficiently large bore to allow rotation of an upright object or patient). Open magnets typically employ two spaced-apart superconductive coil assemblies with the open space between the assemblies allowing for access by medical personnel for surgery or other medical procedures during MRI imaging. The patient may be positioned in that space or also in the bore of the toroidal-shaped coil assemblies. Closed magnets typically have a single, tubular-shaped superconductive coil assembly having a bore. The superconductive coil assembly includes several radially-aligned and longitudinally spaced-apart superconductive main coils each carrying a large, identical electric current in the same direction. The superconductive main coils are thus designed to create a magnetic field of high uniformity within a spherical imaging volume centered within the magnet's bore. A natural choice for a magnet would also be a permanent magnet.

Magnet assembly 12 includes shimming magnets 34 for aligning the non-imaging field with the motion of the charged particles. For example, shimming magnets 34 may include, without limitation, pieces of iron or other magnetic material, or superconductive correction coils, such as Nb—Ti superconductive correction coils. The correction coils may be placed within the superconductive coil assembly radially near and radially inward of the main coils. The shimming magnets are placed close to the trajectory of the charged particles in the non-imaging region. Each correction coil carries a different, but low, electric current in any required direction including a direction opposite to the direction of the electric current carried in the main coils. It is also known to shim a closed magnet by using numerous resistive DC shim coils all located outside the vacuum enclosure (i.e., coil housing) in the bore. The resistive DC shim coils each produce time-constant magnetic fields and may include a single shim coil coaxially aligned with the longitudinal axis and carrying an electric current in a direction opposite to the current direction of the superconductive main coils to correct a harmonic of symmetrical inhomogeneity in the magnetic field within the imaging volume caused by manufacturing tolerances and/or site disturbances.

A collimation controller 36 may be in communication with collimator 28 and MR imager 26 for controlling the operation of collimator 28 in accordance with images from MR imager 26. Collimation controller 36 can cause registration of the radiation beam 30 to the target by using localized target data obtained from MR imager 26 and adjusting the collimator or the collimator leaves such that the radiation beam 30 is steered toward the target.

A position controller 38 may be in communication with positioner 22 and MR imager 26 so as to control the rotational position of object 23 in accordance with images from MR imager 26. Position controller 38 may use localized target data obtained from MR imager 26 to adjust the turntable such that the target is registered with radiation beam 30.

Thus, image-guided radiotherapy system 10 images an upright patient (object 23) and collimates radiation beam 30 with collimator 28, wherein the radiation beam 30 is produced by a motion of charged particles (interacting directly with the patient or converted into a photon beam). The adverse interaction of the magnetic field with the charged particles is substantially eliminated by incorporating shimming magnets 34 to cause co-linearity of the magnetic field and the moving charged particles. Rotating the upright patient on a turntable (positioner 22) enables stereotactic radiation treatment without moving the radiation source 32 or the MRI system.

In accordance with an embodiment of the present invention, collimator 28 or radiation beam modulator 29 may be operable to produce a magnetic field, thus contributing to the main magnetic field. Since the collimator assembly may include a sizable primary collimator for radiation beam 30, the component of the magnetic field produced by the collimator 28 may be significant.

The scope of the present invention includes both combinations and subcombinations of the features described hereinabove as well as modifications and variations thereof which would occur to a person of skill in the art upon reading the foregoing description and which are not in the prior art.

What is claimed is:

1. An image-guided radiotherapy system comprising:
a magnet assembly operable to produce a horizontal imaging field in an imaging region, and a non-imaging field in a non-imaging region;
a positioner operable to rotate an object in said imaging region about a generally vertical rotational axis;
a magnetic resonance (MR) imager in communication with said horizontal imaging field;
a collimator operable to collimate a generally horizontal radiation beam directed towards the object; and
a radiation source operable to produce a radiation beam by moving charged particles substantially parallel to said non-imaging field in said non-imaging region.

2. The system according to claim 1, wherein said magnet assembly comprises shimming magnets for aligning said non-imaging field with motion of said charged particles.

3. The system according to claim 1, wherein said magnet assembly comprises a passageway for said radiation beam.

4. The system according to claim 1, wherein said radiation source is a linear accelerator.

5. The system according to claim 1, wherein said collimator is operable to dynamically shape said radiation beam.

6. The system according to claim 1, further comprising a collimation controller in communication with said collimator and said MR imager.

7. The system according to claim 1, further comprising a position controller in communication with said positioner and said MR imager.

8. The system according to claim 1, wherein said MR imager is operable to reconstruct a 3D image from 2D projections of the object rotated by said positioner.

9. The system according to claim 1, wherein said collimator is operable to produce a magnetic field.

10. The system according to claim 1, wherein said magnet assembly comprises an open magnet system.

11. A method for image-guided radiotherapy comprising:
using a magnet assembly to produce a horizontal imaging field in an imaging region, and a non-imaging field in a non-imaging region;
rotating an object in said imaging region about a generally vertical rotational axis;
collimating a generally horizontal radiation beam of charged particles directed towards the object, the charged particles being substantially parallel to said non-imaging field in said non-imaging region; and
producing MR images of the object making use of said horizontal imaging field produced by said magnet assembly.

12. The method according to claim 11, further comprising aligning said non-imaging field with motion of said charged particles by using shimming magnets.

13. The method according to claim 11, comprising producing said radiation beam with a linear accelerator.

14. The method according to claim 11, further comprising dynamically shaping said radiation beam.

15. The method according to claim 11, further comprising controlling collimation of said radiation beam in accordance with said MR images.

16. The method according to claim 11, further comprising controlling position of the object in accordance with said MR images.

17. The method according to claim 11, further comprising reconstructing a 3D image from 2D projections of the object being rotated.
